(19) United States
(12) Patent Application Publication
(10) Pub. No.: US 2015/0112316 A1
(43) Pub. Date: Apr. 23, 2015

(54) CONTROLLING ACCESS TO AN INTRAVENOUS CATHETER

(71) Applicant: INTERNATIONAL BUSINESS MACHINES CORPORATION, Armonk, NY (US)

(72) Inventors: Gary D. Cudak, Creedmoor, NC (US); Lydia M. Do, Raleigh, NC (US); Christopher J. Hardee, Raleigh, NC (US); Adam Roberts, Moncure, NC (US)

(73) Assignee: INTERNATIONAL BUSINESS MACHINES CORPORATION, Armonk, NY (US)

(21) Appl. No.: 14/060,689

(22) Filed: Oct. 23, 2013

Related U.S. Application Data
(63) Continuation of application No. 14/059,742, filed on Oct. 22, 2013.

(51) Int. Cl. A61M 25/00

(52) U.S. Cl. CPC A61M 25/0043
USPC 604/890.1

(57) ABSTRACT

A method includes logically associating an intravenous catheter and an electronic patient record stored on a computer readable storage medium, wherein the electronic patient record includes medical information specific to an identified patient. An identification code of a syringe to be administered to the identified patient is read in order to identify a liquid contained in the syringe. The blocking member may be electronically actuated to provide fluid communication between a catheter chamber and a catheter line inserted into the identified patient, wherein the fluid communication is provided only while the syringe is received within the catheter chamber and only in response to determining that administration of the identified liquid to the identified patient is compatible within the medical information.

### CROSS-REFERENCE TO RELATED APPLICATIONS

This application is a continuation of U.S. patent application Ser. No. 14/059,742 filed on Oct. 22, 2013, which application is incorporated by reference herein.

### BACKGROUND

1. Field of the Invention

The present invention relates to the use of intravenous catheters, and more particularly relates to systems and methods for introducing fluids to a patient through an intravenous catheter.

2. Background of the Related Art

An intravenous catheter is a medical device that is inserted through the skin into a blood vein and left in place to facilitate the introduction of a fluid or the withdrawal of a blood sample. Depending upon the application, the intravenous catheter may be in continuous or periodic use.

While the intravenous catheter makes these medical interventions simpler and can be used to avoid multiple needle sticks, there is still a potential that the intravenous catheter may be involved in delivery of an inappropriate fluid. Because each patient may have a unique set of conditions and allergies it is important to carefully consider whether a particular fluid, such as an aqueous solution of a medication, is appropriate for a given patient prior to administering the fluid. Furthermore, the large and growing number of medical fluids makes it complex to determine what fluids are appropriate.

While a patient's medical charts and files are a good source of information about what fluids may be appropriate for the patient, the charts themselves must be kept up to date and the person administering fluids must diligently review those charts. Even under the most diligent circumstances imaginable, data regarding potential drug interactions may become available at any time. Unless the person administering or prescribing the fluids has received this information, the patient may not benefit from this recent data. Still further, some fluids should only be administered upon verifying the patient's current condition, such as heart rate or blood pressure.

### BRIEF SUMMARY

One embodiment of the present invention provides a method, comprising: logically associating an intravenous catheter and an electronic patient record stored on a computer readable storage medium, wherein the electronic patient record includes medical information specific to an identified patient; reading an identification code of a syringe to be administered to the identified patient, wherein the identification code identifies a liquid contained in the syringe; and electronically actuating a blocking member to provide fluid communication between a catheter chamber and a catheter line inserted into the identified patient, wherein the fluid communication is provided only while the syringe is received within the catheter chamber and only in response to determining that administration of the identified liquid to the identified patient is compatible within the medical information specific to the identified patient.

### BRIEF DESCRIPTION OF THE SEVERAL VIEWS OF THE DRAWINGS

FIG. 1 is a diagram including the basic components of an access control system for an intravenous catheter.

FIG. 2 is a diagram of the access control system with a syringe inserted into the catheter chamber.

FIG. 3 is a diagram of the access control system with the blocking mechanism in an unblocked position.

FIG. 4 is a diagram of the access control system with a tip of the syringe extending through the blocking mechanism.

FIG. 5 is a diagram of the access control system with the syringe plunger depressed to deliver a fluid into the catheter.

FIG. 6 is a partial cross-sectional side view of the catheter chamber receiving a syringe with the syringe plunger fully depressed.

FIG. 7 is a diagram of a computer that may be used in the access control system in accordance with one embodiment of the invention.

FIG. 8 is a flowchart of a method of controlling access to a catheter in accordance with another embodiment of the invention.

### DETAILED DESCRIPTION

One embodiment of the present invention provides a system, comprising: an intravenous catheter having a catheter chamber, a catheter line for insertion into a blood vessel of the identified patient, and an electronically actuatable blocking member providing selective fluid communication between the catheter chamber and the catheter line; a reader secured to the catheter chamber for reading an identification code of a syringe received within the catheter chamber, wherein the identification code identifies a liquid contained in the syringe; and a control system having access to a computer readable storage medium storing an electronic patient record that includes medical information specific to an identified patient, wherein the control system is in communication with the reader and runs an application program logically associating the intravenous catheter and the medical information specific to the identified patient, determining whether the administration of the identified liquid to the identified patient is compatible within the medical information specific to the identified patient, and controlling electronic actuation of the blocking member.

The application program may, for example, send a signal to electronically actuate the blocking member, such as a valve coupled to an electronic actuator, to provide fluid communication between the catheter chamber and the catheter line only while the syringe is received within the catheter chamber and only in response to determining that administration of the identified liquid to the identified patient is compatible within the medical information specific to the identified patient.

Embodiments of the system may include a reader selected from an optical scanner, a near field communication (NFC) antennae, or a small contact smart card reader. Where the reader is an optical scanner, the syringe may include a label that is secured to or printed on the syringe. A printed identification code may be a bar code or a quick response (QR) code. Optionally, the reader may be in wireless communication with the control system.

The medical information specific to the identified patient includes data selected from patient drug history, patient allergies, patient diet. A doctor may also place an entry into the medical information specific to the identified patient, wherein the entry blocks a specific medication, a family of medications or everything for a patient without prior approval. Furthermore, in certain circumstances one or more medications can be placed on a blocked list for the entire facility. The term “electronic patient record” should be interpreted in a generic sense, and does not imply a record that is consistent with any industry standards or specifications.

In a still further embodiment, the control system has further access to data selected from drug interactions, and food and drug interactions. Optionally, the application program may send a signal to electronically actuate the blocking member to provide fluid communication between the catheter chamber and the catheter line only in response to determining that administration of the identified liquid to the identified patient will not cause a prohibited drug interaction or food and drug interaction with drugs previously administered to the identified patient as listed in the medical information specific to the identified patient.

In yet another embodiment, the control system may have further access to treatment data identifying fluids that are approved for various patient conditions. Optionally, the application program may send a signal to electronically actuate the blocking member to provide fluid communication between the catheter chamber and the catheter line only in response to determining that the treatment data indicates that the identified liquid is approved for treatment of one or more condition listed in the medical information specific to the identified patient.

The system may, in one or more embodiments, further comprise a sensor for detecting that a plunger within the syringe has reached a depressed position, wherein the sensor is in communication with the control system. For example, the sensor may detected the plunger position using a first set of electronic contacts extending through a wall of the syringe and a second set of electronic contacts extending through a wall of the catheter. If the sensor detects that the plunger within the syringe has reached a depressed position, the fact that the identified fluid was administered may be stored in the electronic patient record for the identified patient. Such stored information may be considered later in determining whether any subsequent fluid is appropriate to be administered to the identified patient. Preferably, the stored information will also identify the day and time and/or other details about the fluid administration.

Although the embodiments described above are referred to as intravenous catheters, it should be recognized that the invention may include the mechanism of the catheter chamber in a split line that is some distance apart from the catheter line.

Another embodiment of the present invention provides a method, comprising: logically associating an intravenous catheter and an electronic patient record stored on a computer readable storage medium, wherein the electronic patient record includes medical information specific to an identified patient; reading an identification code of a syringe to be administered to the identified patient, wherein the identification code identifies a liquid contained in the syringe; and electronically actuating a blocking member to provide fluid communication between a catheter chamber and a catheter line inserted into the identified patient, wherein the fluid communication is provided only while the syringe is received within the catheter chamber and only in response to determining that administration of the identified liquid to the identified patient is compatible within the medical information specific to the identified patient.

Logically associating an electronic patient record and an intravenous catheter may include, for example, storing a logical association between a patient identification code and an electronically readable serial number of the catheter. An example of a patient identification code may be a patient's name, a patient's social security number, or some uniquely assigned identification code that only has significance within a particular medical facility. For example, a “logical association” might be established by storing the catheter ID and the patient ID in a common record of a list (i.e., a row of a table), or by storing the catheter ID and the patient ID in separate records using a common reference ID (as in a relational database). The logical association between two identifiers (IDs), such as the catheter ID and the patient ID, can be established using any data storage or programming technique that represents the physical condition that the catheter having the catheter ID has been physically inserted in the patient having the patient ID. As a result, the methods of the present invention recognize that the contents of a syringe read by the catheter (having the catheter ID) are being presented for administration to the associated patient (having the patient ID).

Reading an identification code of a syringe received within the catheter chamber may, in one embodiment, include optically scanning an identification code that is optically detectable on an outwardly facing surface of the syringe. Accordingly, the catheter may include an optical scanning device secured to the catheter chamber for optically scanning the identification code. Such an optical scanning device is preferably in wired or wireless communication with a control system that has access to the electronic patient record. For example, the control system may run an application program that determines whether the administration of the identified liquid to the identified patient is compatible within the medical information specific to the identified patient.

Optionally, the application program may generate an alert signal in response to determining that administration of the identified liquid to the identified patient is not compatible within the medical information specific to the identified patient. Such an alert may be audible and/or visual to the person attempting the administration and/or other medical facility personnel. Preferably, a record of the attempted administration of the identified liquid is stored in the electronic patient record.

Embodiments of the method may further include detecting that the fluid has been dispensed from the syringe while the syringe is received within the catheter chamber, such as by detecting that a plunger within the syringe reaches a depressed position. If it is detected that the fluid has been dispensed while so positioned, the method may store data in the electronic patient record for the identified patient indicating that the identified fluid was administered. Preferably, the stored data will further include a day and time at which the identified fluid was administered.

Measures of the patient's vital signs, blood chemistry or other data can be manually or automatically input and used in the determination of whether to unblock the administration of a fluid. For instance, it is important to measure the patient's heart rate prior to administering digoxin. For example, the heart rate may need to be at least 60 beats per minute.

It should be recognized that any one or more system embodiment of the present invention may be incorporated into any one or more method embodiment of the present invention, and vice versa. Furthermore, features or limitations of any one embodiment may be combined with the features or limitations of any other embodiment described herein, unless the context would clearly prohibit such combination. Accordingly, this disclosure of the invention should be read as a whole to determine the full scope of the embodiments of the present invention.

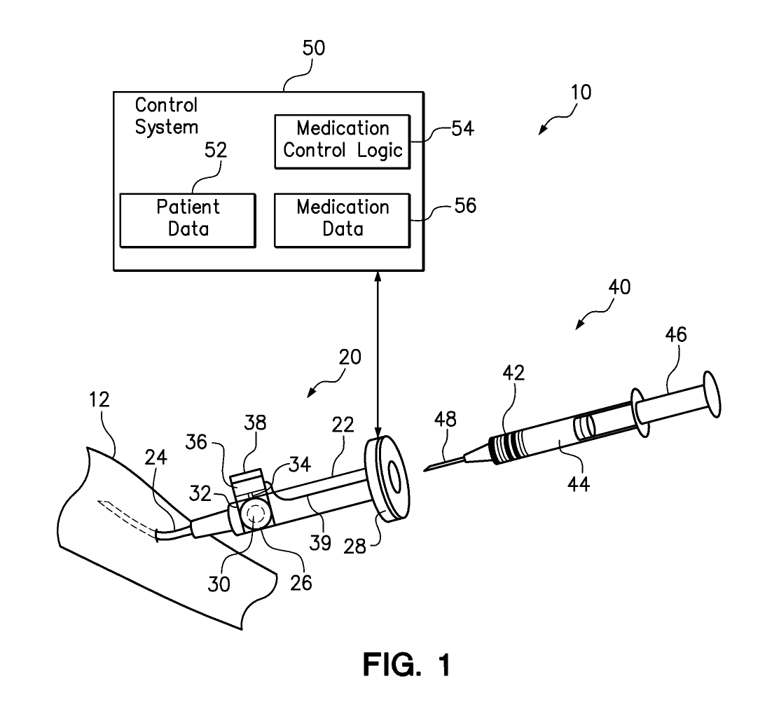

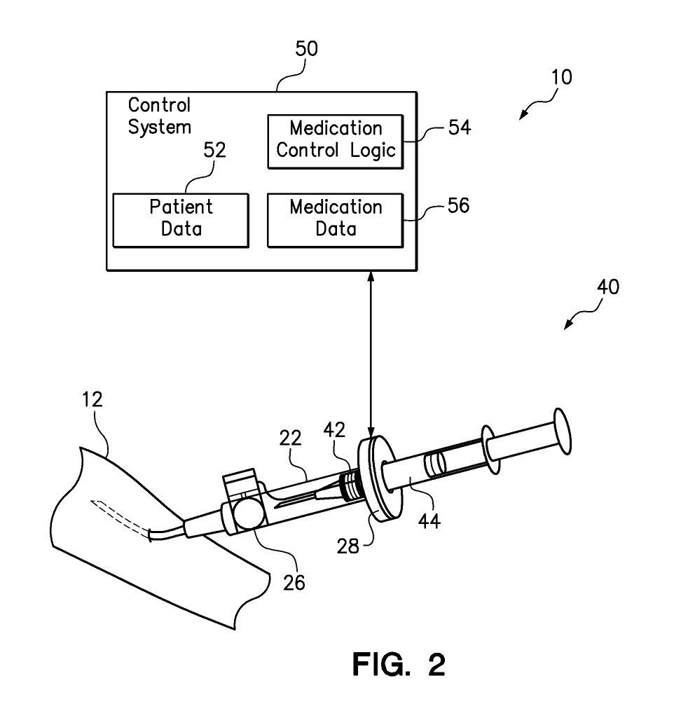

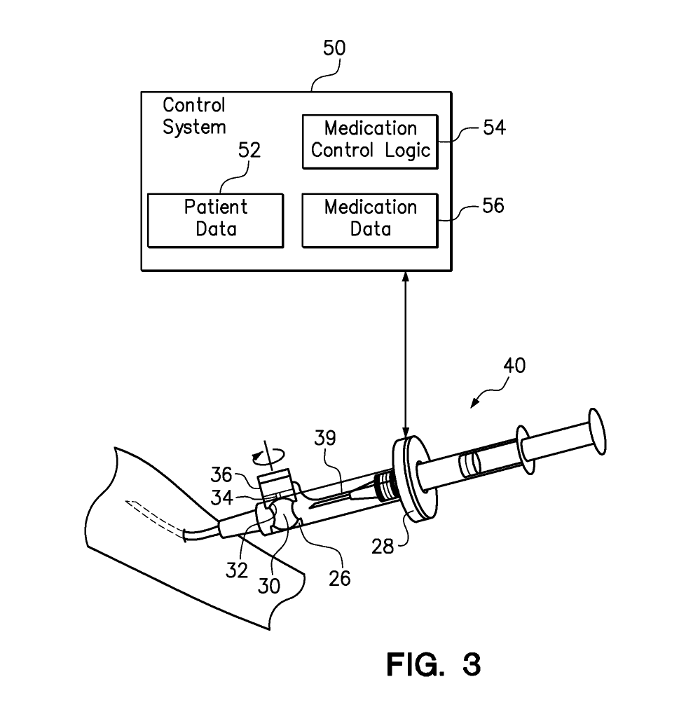

FIG. 1 is a diagram including the basic components of an access control system for an intravenous catheter. The system 10 includes an intravenous catheter 20 having a catheter chamber 22, a catheter line 24 for insertion into a blood vessel of the identified patient (see patient's arm 12), and an electronically actuatable blocking member 26 providing selective fluid communication between the catheter chamber 22 and the catheter line 24. As shown, the blocking member 26 is a ball valve include a ball 30, a seat 32 that seals around the ball, and a shaft 34 coupled to the ball 30 to facilitate rotation of the ball between the closed (blocked) position of FIGS. 1-2 and an open (unblocked) position shown in FIGS. 3-5. An electronic actuator 36, such as a motor, is coupled to the shaft 34 and receives power from a power source 38, such as a battery.

A reader 28 is secured to the catheter chamber 22 for reading an identification code 42 of a syringe 40 that is receivable within the catheter chamber 22. The identification code 42 identifies a liquid 44 contained in the syringe 40. At the appropriate time and in the appropriate position, a plunger 46 may be manually depressed to push the liquid 44 out of the syringe through a needle 48 or an alternative tip, such as a Luer lock.

A control system 50 has access to a computer readable storage medium storing an electronic patient record 52 that includes medical information specific to an identified patient. The control system 50 is in communication with the reader 28 or a microcontroller associated with the reader. In use, the control system 50 and runs an application program, such as the medication control logic 54. The application 54 logically associates the intravenous catheter 20 and the medical information 52 (such as a record of a patient information database) that is specific to the identified patient 12. This logical association may take place before or during the first use of the catheter 20. The application 54 may then be responsible for determining whether the administration of the identified liquid 44 to the identified patient 12 is compatible within the medical information 52 specific to the identified patient. In certain embodiments, the control system 50 includes medication data 56 that identifies drug interactions (or food and drug interactions) and/or treatment data. Combinations of drugs that may cause drug interactions should be avoided, such that the blocking member will remain blocked to prevent administration of a drug that interacts with a previously administered drug. Treatment data my include lists of drugs and the conditions or diagnosis that are appropriately treated with each drug. If the patient record shows a particular condition, the control system may unblock the blocking member for the administration of a drug that is identified in the treatment data as being appropriate for that particular condition, absent a drug interaction conflict. Optionally, the control system will only allow administration of a list of drugs specifically associated with the medical information stored for the identified patient.

If the identified liquid is found to be compatible or otherwise approved, then the control system may send a signal to control electronic actuation of the blocking member 26 from a blocked position to an unblocked position. Such a signal may be communicated through the reader or associated microcontroller, and relayed through a wire 39 to the actuator 36.

FIG. 2 is a diagram of the access control system 10 with the syringe 40 inserted into the catheter chamber 22. In this position, or as the syringe passes through to this position, the reader 28 can read the identification code 42 that identifies a liquid 44 contained in the syringe 40. The identification code 42 is transmitted from the reader 28 to the control system 50 for use by the medication control logic 54 in determining whether or not to unblock the blocking member 26.

FIG. 3 is a diagram of the access control system 10 with the blocking mechanism 26 in an unblocked position. To get into this position, the control system 50 has sent a signal to the reader 28, which conveyed the signal via the wire 39 to the actuator 36. The actuator 36 then turns the shaft 34 (typically 90 degrees) so that a hole through the ball 30 aligns with openings in the seat 32, which is also aligned with the tip of the syringe 40.

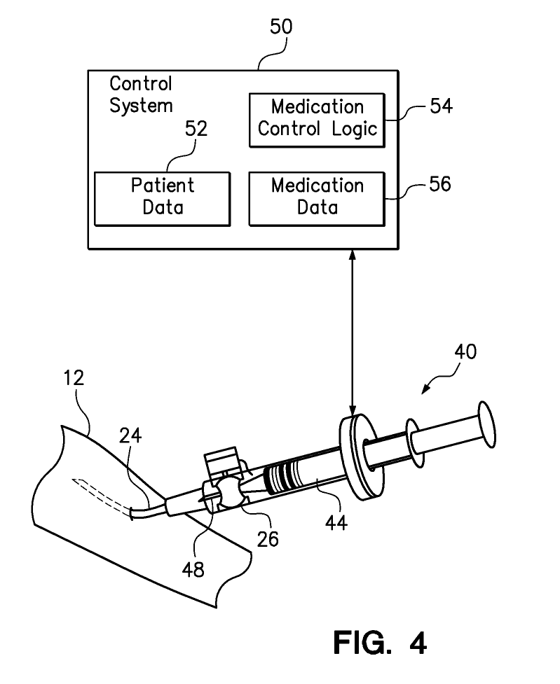

FIG. 4 is a diagram of the access control system 10 with a tip or needle 48 of the syringe 40 extending through the blocking mechanism 26. At this point, there is nothing to prevent the administration of the identified fluid 44 into the catheter line 24 and into the patient 12.

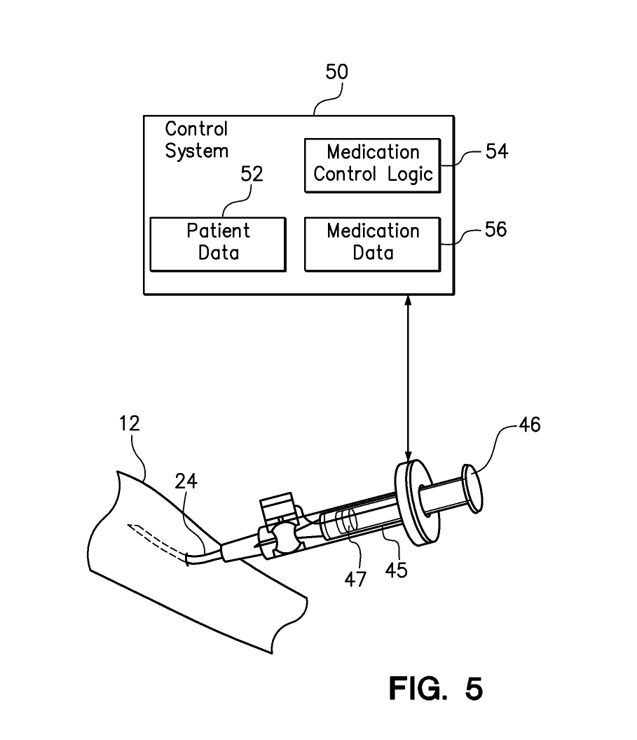

FIG. 5 is a diagram of the access control system 10 with the syringe plunger 46 depressed to deliver the fluid into the catheter line 24. Note that a distal end 47 of the plunger 46 is positioned at the end of a barrel 45 the syringe 40.

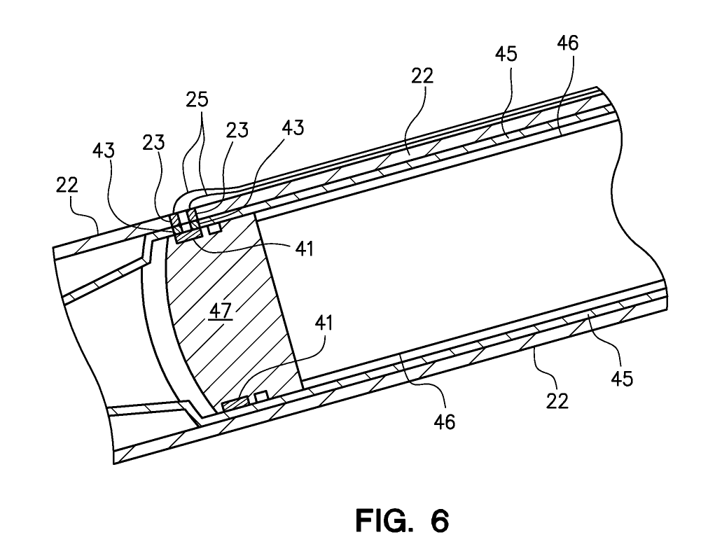

FIG. 6 is a partial cross-sectional side view of the catheter chamber 22 receiving the syringe barrel 45 with the syringe plunger 46 fully depressed. In one embodiment, a sensor (not shown; optionally part of the circuitry in the reader 28) detects the depressed position of the plunger 46. The end 47 of the plunger 46 has a conductive metal band 41 that may extend around the circumference of the end 47. Accordingly, with the plunger 46 in the fully depressed position shown, the metal band 41 comes into lateral alignment with a pair of metal contacts 43 that extend through the wall of the syringe barrel 45. Each of these metal contacts 43 is also laterally aligned with a pair of metal contacts 23 that extend through the wall of the catheter chamber 22, such that the metal band 41 completes a circuit that includes a pair of wires 25. The pair of wires 25 are shown extending along the catheter chamber 22 to a sensor packaged with the reader.

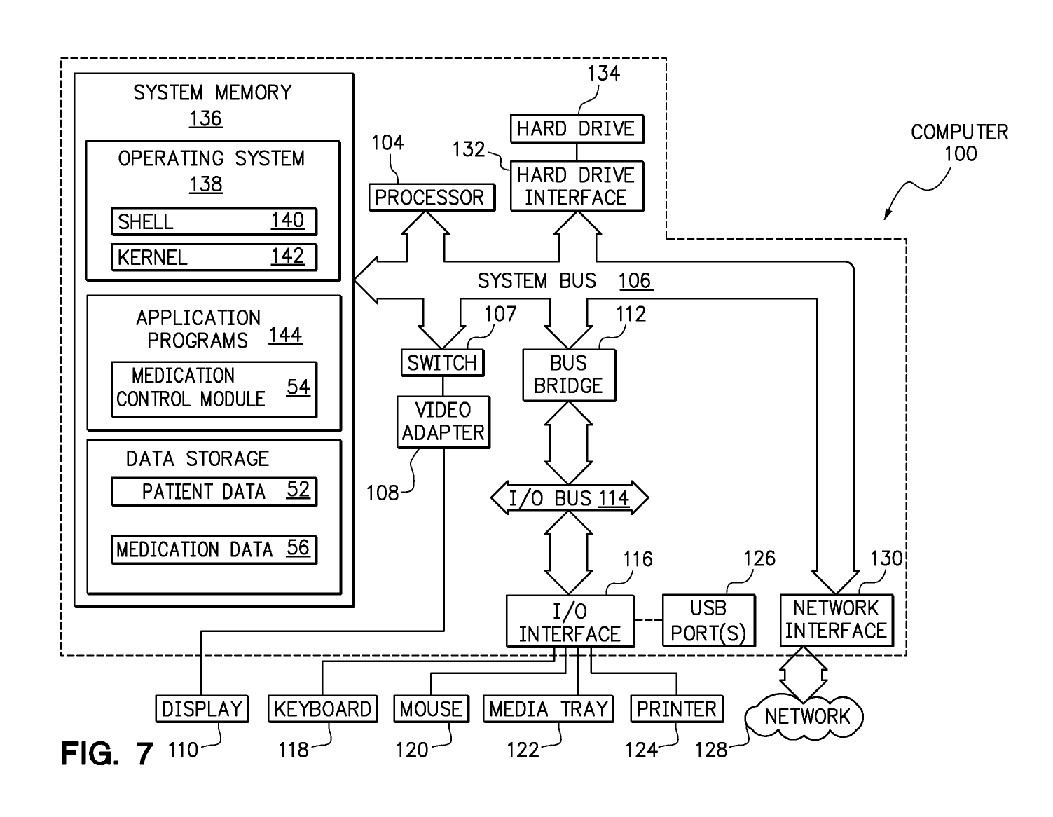

FIG. 7 is a diagram of a computer 100 that may be used in the access control system in accordance with one embodiment of the invention. Optionally, the computer 100 may be all or part of the control system 50 (See FIGS. 1-5).

Computer 100 includes a processor unit 104 that is coupled to a system bus 106. Processor unit 104 may utilize one or more processors, each of which has one or more processor cores. A video adapter 108, which drives/supports a display 110, is also coupled to system bus 106. In one embodiment, a switch 107 couples the video adapter 108 to the system bus 106. Alternatively, the switch 107 may couple the video adapter 108 to the display 110. In either embodiment, the switch 107 is a switch, preferably mechanical, that allows the display 110 to be coupled to the system bus 106, and thus to be functional only upon execution of instructions that support the processes described herein.

System bus 106 is coupled via a bus bridge 112 to an input/output (I/O) bus 114. An I/O interface 116 is coupled to I/O bus 114. I/O interface 116 affords communication with various I/O devices, including a keyboard 118, a mouse 120, a media tray 122 (which may include storage devices such as CD-ROM drives, multi-media interfaces, etc.), a printer 124, and external USB port(s) 126. While the format of the ports connected to I/O interface 116 may be any known to those skilled in the art of computer architecture, in a preferred embodiment some or all of these ports are universal serial bus (USB) ports.

As depicted, the computer 100 is able to communicate over a network 128 using a network interface 130. Network 128 may be an external network such as the Internet, or an internal network such as an Ethernet or a virtual private network (VPN).

A hard drive interface 132 is also coupled to system bus 106. Hard drive interface 132 interfaces with a hard drive 134. In a preferred embodiment, hard drive 134 populates a system memory 136, which is also coupled to system bus 106. System memory is defined as a lowest level of volatile memory in computer 100. This volatile memory includes additional higher levels of volatile memory (not shown), including, but not limited to, cache memory, registers and buffers. Data that populates system memory 136 includes computer 100's operating system (OS) 138 and application programs 144.

The operating system 138 includes a shell 140, for providing transparent user access to resources such as application programs 144. Generally, shell 140 is a program that provides an interpreter and an interface between the user and the operating system. More specifically, shell 140 executes commands that are entered into a command line user interface or from a file. Thus, shell 140, also called a command processor, is generally the highest level of the operating system software hierarchy and serves as a command interpreter. The shell provides a system prompt, interprets commands entered by keyboard, mouse, or other user input media, and sends the interpreted command(s) to the appropriate lower levels of the operating system (e.g., a kernel 142) for processing. Note that while shell 140 is a text-based, line-oriented user interface, the present invention will equally well support other user interface modes, such as graphical, voice, gestural, etc.

As depicted, the OS 138 also includes kernel 142, which includes lower levels of functionality for OS 138, including providing essential services required by other parts of OS 138 and application programs 144, including the medication control logic module 54. The system memory 136 of computer 100, or the hard disk drive 134 may store the patient data 52 and the medication data 56.

The hardware elements depicted in computer 100 are not intended to be exhaustive, but rather are representative components suitable for use in accordance with the present invention. For instance, computer 100 may include alternate memory storage devices such as magnetic cassettes, digital versatile disks (DVDs), Bernoulli cartridges, and the like. These and other variations are intended to be within the spirit and scope of the present invention.

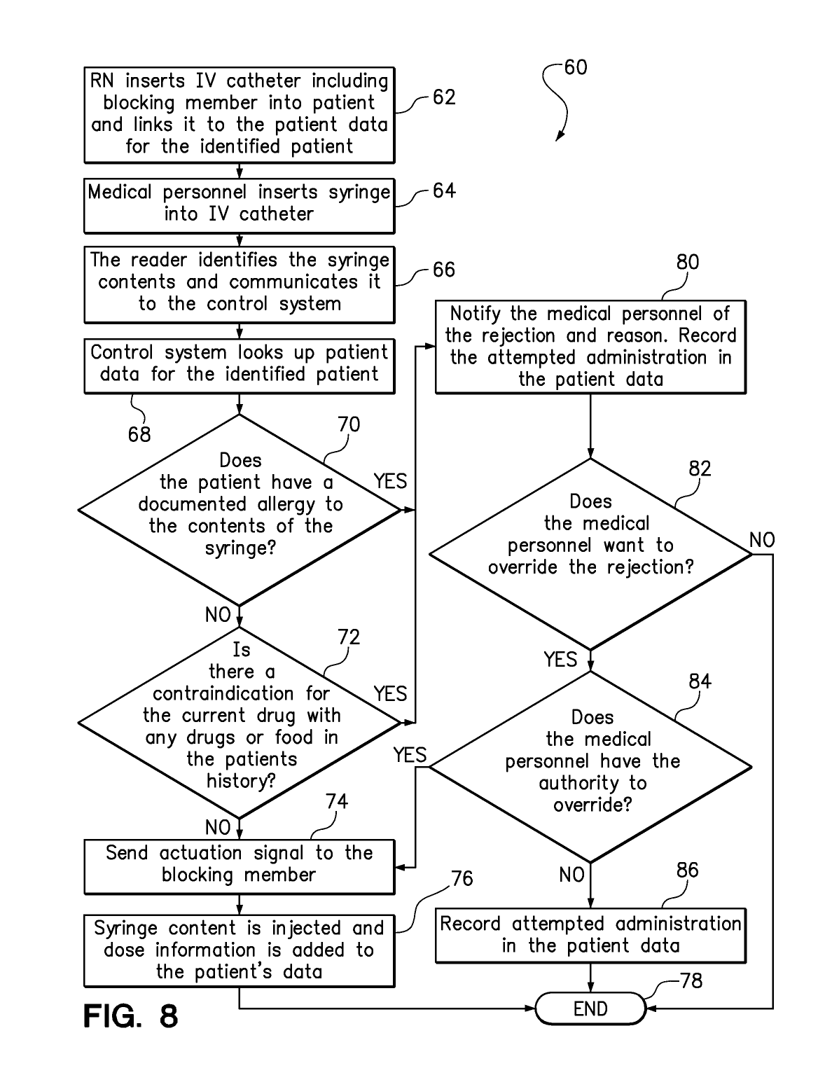

FIG. 8 is a flowchart of a method 60 for controlling access to a catheter in accordance with another embodiment of the invention. In step 62, a registered nurse (RN) or other medical personnel inserts an intravenous catheter of the present invention into a patient and links or logically associates the intravenous catheter and the patient data for the identified patient. In step 64, the medical personnel inserts a syringe into the catheter chamber of the intravenous catheter. The syringe may have a needle, Luer lock or a line from a bag of intravenous fluid, and the syringe is presumably filled with a liquid that the medical personnel intends to administer to the patient. The blocking member that is part of the catheter remains blocked by default, but a reader of the catheter is now able to read an identification code on the syringe. In step 66, the reader identifies the syringe contents and communicates that information to the control system. Then, in step 68, the control system looks up the patient data for the identified patient.

Step 70 determines whether the patient data for the identified patient (“the patient”) shows an allergy to the contents of the syringe (“the identified fluid”), and step 72 determines whether there is a contraindication for the current drug (“the identified fluid”) with any drugs or food in the patient's history (“the patient data”). If both of steps 70 and 72 return negative, then step 74 sends an actuation signal to the blocking member so that the blocking member moves into the unblocked position. With the blocking member unblocked, the syringe gains access to the catheter line. Accordingly, in step 76, the syringe contents are injected and the dose information is preferably added to the patient data, before the process ends in step 78.

However, if either of steps 70 or 72 return positive, then the process proceeds to step 80, wherein medical personnel are notified that administration of the syringe fluid has been rejected and the reason therefore. Preferably, the attempted administration is recorded in the patient data for the identified patient.

In a further embodiment, the method proceeds to steps 82 and 84 to implement an override option. In step 82, the medical personnel is prompted to identify whether or not they want to override the rejection. If not, the method ends in step 78. However, if the medical personnel wants to override the rejection, then step 84 determines whether the medical personnel has the authority to execute the override. Such a determination may require a medical personnel identification and password, and a predetermined authorization level for each of the medical personnel. If the override is authorized, then the method returns to step 74 in order to send an actuation signal to the blocking member, such that the syringe contents may be subsequently administered. In the authority is not presented, then step 86 records the attempted administration in the patient data before the method ends in step 78.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing. Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention may be described with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, and/or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, components and/or groups, but do not preclude the presence or addition of one or more other features, integers, steps, operations, elements, components, and/or groups thereof. The terms “preferably,” “preferred,” “prefer,” “optionally,” “may,” and similar terms are used to indicate that an item, condition or step being referred to is an optional (not required) feature of the invention.

The corresponding structures, materials, acts, and equivalents of all means or steps plus function elements in the claims below are intended to include any structure, material, or act for performing the function in combination with other claimed elements as specifically claimed. The description of the present invention has been presented for purposes of illustration and description, but it is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A method, comprising:
logically associating an intravenous catheter and an electronic patient record stored on a computer readable storage medium, wherein the electronic patient record includes medical information specific to an identified patient;
reading an identification code of a syringe to be administered to the identified patient, wherein the identification code identifies a liquid contained in the syringe; and
electronically actuating a blocking member to provide fluid communication between a catheter chamber and a catheter line inserted into the identified patient, wherein the fluid communication is provided only while the syringe is received within the catheter chamber and only in response to determining that administration of the identified liquid to the identified patient is compatible within the medical information specific to the identified patient.

2. The method of claim 1, wherein logically associating an electronic patient record and an intravenous catheter, includes storing a logical association between a patient identification code and an electronically readable serial number of the catheter.

3. The method of claim 1, wherein the electronically actuatable blocking member is a valve coupled to an electronic actuator.

4. The method of claim 1, wherein reading an identification code of a syringe received within the catheter chamber, includes optically scanning an identification code that is optically detectable on an outwardly facing surface of the syringe

5. The method of claim 4, wherein the catheter further includes an optical scanning device secured to the catheter chamber for optically scanning the identification code.

6. The method of claim 5, wherein the optical scanning device is in wired or wireless communication with a computer that has access to the electronic patient record.

7. The method of claim 6, wherein the computer runs an application program that determines whether the administration of the identified liquid to the identified patient is compatible within the medical information specific to the identified patient.

8. The method of claim 7, wherein the application program, only in response to determining that administration of the identified liquid to the identified patient is compatible within the medical information specific to the identified patient, sends a signal to electronically actuate the blocking member to provide fluid communication between the catheter chamber and the catheter line.

9. The method of claim 8, wherein the medical information specific to the identified patient includes data selected from patient drug history, patient allergies, patient diet.

10. The method of claim 7, wherein the application program, in response to determining that administration of the identified liquid to the identified patient is not compatible within the medical information specific to the identified patient, generates an alert signal.

11. The method of claim 10, further comprising:
storing a record of the attempted administration of the identified liquid.

12. The method of claim 7, wherein the computer has further access to data selected from drug interactions, and food and drug interactions.

13. The method of claim 12, wherein the application program, only in response to determining that administration of the identified liquid to the identified patient will not cause a prohibited drug interaction or food and drug interaction with drugs previously administered to the identified patient as listed in the medical information specific to the identified patient, sends a signal to electronically actuate the blocking member to provide fluid communication between the catheter chamber and the catheter line.

14. The method of claim 7, wherein the computer has further access to treatment data identifying fluids that are approved for various patient conditions.

15. The method of claim 7, wherein the application program, only in response to determining that the treatment data indicates that the identified liquid is approved for treatment of one or more condition listed in the medical information specific to the identified patient, sends a signal to electronically actuate the blocking member to provide fluid communication between the catheter chamber and the catheter line.

16. The method of claim 1, wherein enabling the blocking member to provide fluid communication between the catheter chamber and the catheter line, includes transmitting an electronic signal to the electronically actuatable blocking member.

17. The method of claim 1, further comprising:
detecting that the fluid has been dispensed from the syringe while the syringe is received within the catheter chamber.

18. The method of claim 17, wherein detecting that the fluid has been dispensed from the syringe while the syringe is received within the catheter chamber, includes detecting a plunger within the syringe reaches a depressed position.

19. The method of claim 17, further comprising:
storing, in the electronic patient record for the identified patient, that the identified fluid was administered.

20. The method of claim 17, further comprising:
storing, in the electronic patient record for the identified patient, that the identified fluid was administered and a time at which the identified fluid was administered.
